(19) United States
(12) Patent Application Publication
(10) Pub. No.: US 2025/0107743 A1
(43) Pub. Date: Apr. 3, 2025

(54) METHODS OF DOCUMENTING AND TREATING SYMPATHETIC AND PARASYMPATHETIC (AUTONOMIC) DYSFUNCTION

(71) Applicant: Physio PS, Inc., Richboro, PA (US)

(72) Inventor: Joseph Colombo, Richboro, PA (US)

(73) Assignee: Physio PS, Inc., Richboro, PA (US)

(21) Appl. No.: 18/905,682

(22) Filed: Oct. 3, 2024

Related U.S. Application Data
(60) Provisional application No. 63/542,171, filed on Oct. 3, 2023.

(51) Int. Cl. A61B 5/00; A61B 5/0205; A61B 5/024; A61B 5/08

(52) U.S. Cl. CPC A61B 5/4035; A61B 5/0205; A61B 5/7275; A61B 5/024; A61B 5/08; A61B 5/4884; A61B 2503/08

(57) ABSTRACT

Diagnostic methods for assessing the parasympathetic and sympathetic nervous systems separately and simultaneously are disclosed. These methods involve collecting pulse and respiratory data during a series of repeated, escalating challenges, and plotting the data on a time series utilizing signal processing. From this data, parasympathetic excess (PE), sympathetic excess (SE), and alpha-sympathetic withdrawal (SW) can be determined, and several conditions diagnosed and treated based on the observation of PE, SE, and/or SW during various stages of the challenges.

### REFERENCE TO RELATED APPLICATIONS

This is a utility patent application claiming priority and benefit to Provisional Application No. 63/542,171, filed 3 Oct. 2023, also entitled “Methods of Documenting and Treating Sympathetic and Parasympathetic (Autonomic) Dysfunction.” The contents of the above-referenced application are fully incorporated by reference.

### FIELD OF THE APPLICATION

The invention relates to methods for preventing, reducing or reversing nervous system dysfunction in a multitude of subjects, from a multitude of fields and sub-fields in medicine, both physiologic and psychologic, chronic care and critical care, and in a multitude of applications outside the field of medicine where measuring human responses to protect and promote health is required.

### BACKGROUND

The Autonomic Nervous System (ANS) is comprised of two branches: the Parasympathetic and Sympathetic (P&S) nervous systems. The P&S nervous systems control and coordinate virtually every cell in the human body. Their primary function is to maintain homeostasis under all conditions. Together they do so by coordinating and maintaining normal organ or organ system function in response to stresses, both healthy and unhealthy; including physical, physiological, mental, psychological, genetic, social, and environmental stresses. The P&S systems do so even when they themselves are disordered, to a point. As a result, P&S function (both individual and coordinated) and balance (known as Sympathovagal Balance, or SB, reflecting the relationship between P&S activity).

The sympathetic (or adrenergic) nervous system controls and coordinates adrenaline responses. It is typically referred to as the fight or flight nervous system, and is intended for short-term responses. Prolonged or sustained sympathetic reactions, as well as excessive or insufficient responses, indicate disorder or disease. The sympathetic system reacts to the thresholds established by the parasympathetic system, and uniquely (directly) controls the vasculature. It typically increases metabolism and responds to stresses. The sympathetic is the slower system to respond to a stimulus; typically responding within three to five heart-beats.

The parasympathetic (or cholinergic) nervous system is typically referred to as the “rest and digest” nervous system. It is also the “protective” system. The parasympathetic sets the metabolic threshold around which the sympathetic reacts. Excessive or insufficient Parasympathetic reactions may also indicate disorder or disease. The parasympathetic system uniquely controls and coordinate (directly) the immune, histamine and the gastrointestinal systems. It typically decreases metabolism, increases GI motility, coordinates immune responses, and supports proper tissue perfusion throughout the body. The parasympathetic is the faster system to respond to a stimulus; typically responding within one to three heart-beats.

These two branches are hardly ever at rest, and when a person is resting, they are arguably the most active. Therefore, they must be tested both at rest (a challenge) and in response to various challenges, both individually and collectively, to be fully assessed. Both P&S responses must be observed independently (as in mathematical independence) and simultaneously during all of these challenges to be fully assessed. The standard challenges for the P&S nervous systems are: (1) resting; (2) slow paced breathing at 6 breaths per minute to challenge the parasympathetic nervous system, specifically; (3) Short Valsalva maneuvers of less than or equal to 15 seconds to challenge the sympathetic nervous system, specifically; (4) a positive, head-up, postural change challenge (typically from sitting to standing) to challenge the coordination of both systems as well as to act as a tilt table test.

The tilt-table test is currently considered to be the standard challenge for the autonomic nervous system to assess causes of lightheadedness and dizziness. However, the tilt-table does this poorly, its results are often inconclusive or misleading, and it cannot differentiate parasympathetic from sympathetic activity, it merely reports on symptoms which oftentimes must be artificially induced. Furthermore, the tilt-table is not a natural maneuver, and therefore in and of itself is artificial.

U.S. Pat. No. 7,079,888 (the contents of which are incorporated herein by reference) describes a non-invasive method of simultaneously monitoring the sympathetic and parasympathetic nervous systems during these and other challenges, utilizing continuous wavelet transformation of pulse and respiratory signals to obtain a ratio between the low-frequency areas and respiratory-frequency areas, representing the sympathovagal balance.

Since the '888 Patent, which was concerned primarily with short-term conditions which could be diagnosed on the order of 15 minutes (e.g., Paradoxical Parasympathetics), it has been discovered that more long-term utilizations of this and other methods of monitoring the sympathetic/parasympathetic systems, enable new autonomic dysfunctions are able to be identified from the unfiltered EKG waveform, including Parasympathetic Excess (PE) and alpha-Sympathetic Withdrawal (SW). Also, formerly known autonomic dysfunctions may now be documented, including beta-Sympathetic Excess (SE), which can be differentiated from SW and PE (and SW and PE differentiated from each other). Formerly known risk factors may now be documented earlier and more specifically, such as Small Fiber Disease (SFD), Advanced Autonomic Dysfunction (AAD), Diabetic Autonomic Neuropathy (DAN), Cardiovascular Autonomic Neuropathy (CAN), high morbidity risk (including risk of Long-COVID), mortality risk (including risk of Major Adverse Cardiovascular Events, or MACE), risk of depression and depression-related disorders, risk of falling, risk of repeat addiction (including Opioid addiction). With these abnormal conditions documented, they are now treatable, thereby reducing medication-load, hospitalizations, and re-hospitalizations, ultimately reducing health care costs for the individual and for the nation.

Two key presentations discernible with this monitoring are Postural Orthostatic Tachycardia Syndrome (POTS) and Vasovagal Syncope (VVS). Both are causes of lightheadedness, and if both are not documented correctly and treated simultaneously, the patient will not be relieved of the symptoms, which if prolonged, often become disabling. Currently, POTS and VVS may not be differentiated with standard means of measurement, including tilt-table testing with beat-to-beat blood pressure or heart rate variability. As a result, the current clinical thinking is to only treat symptoms because the cause is unknown.

A need therefore exists for a method of cardiorespiratory monitoring and diagnosis which can provide additional information to the clinician, enabling the component symptoms of POTS/VVS to be isolated and differentiated, and the SW, tachycardia, PE and SE to be individually treatable.

Methods disclosed in the detailed description below meet this need.

### DRAWINGS

FIG. 1 shows a high-level flow chart of the method based on respiratory and cardiovascular data from a patient.

FIG. 2 shows a flow chart of an embodiment of a testing procedure to gather the data used in FIG. 1.

FIG. 3 shows “normal” trends plots for a variety of ages.

FIG. 4 shows the “normal” ranges of sympathetic and parasympathetic activity for various stages of the testing in FIG. 2.

FIG. 4A shows a chart of expected parasympathetic response analysis from FIG. 4.

FIG. 5 shows various interpretations of the signals which can be extracted from the inventive method.

FIG. 6 shows an exemplar processing device for implementing the inventive methods.

The detailed description below is described with reference to the above drawings.

### DESCRIPTION OF THE INVENTION

Before describing selected embodiments of the present disclosure in detail, it is to be understood that the present invention is not limited to the particular embodiments described herein. The disclosure and description herein is illustrative and explanatory of one or more presently preferred embodiments and variations thereof, and it will be appreciated by those skilled in the art that various changes in the design, organization, order of operation, means of operation, equipment structures and location, methodology, and use of mechanical or electronic fIequivalents may be made without departing from the spirit of the invention.

As well, it should be understood that the drawings are intended to illustrate and plainly disclose presently preferred embodiments to one of skill in the art, but are not intended to be manufacturing level drawings or renditions of final products and may include simplified conceptual views as desired for easier and quicker understanding or explanation. As well, the relative size and arrangement of the components may differ from that shown and still operate within the spirit of the invention. Because many varying and different embodiments may be practiced within the scope of the concepts herein taught, and because many modifications may be made in the embodiments described herein, it is to be understood that the details herein are to be interpreted as illustrative and non-limiting.

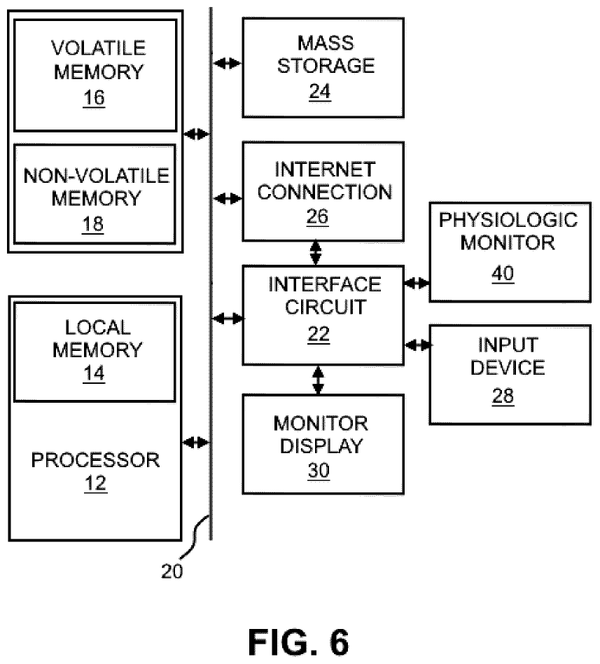

Turning first to FIG. 6, a schematic view of at least a portion of an example implementation of a processing device 10 is shown, according to one or more aspects of the present disclosure. The processing device 10 may be in communication with at least one physiologic monitor 40, from which it receives patient inputs, at least one input device 28, from which it receives clinician inputs, and at least one monitor display 30, to which it sends outputs.

The processing device 10 may comprise, for example, one or more processors, special-purpose computing devices, servers, personal computers (e.g., desktop, laptop, and/or tablet computers), PDA devices, smartphones, internet appliances, and other types of computing devices. The processing device 10 may comprise a processor 12, including a general-purpose programmable processor, local memory 14, volatile memory 16, and non-volatile memory 18. The processor 12 may execute, among other things, any machine-readable coded instructions and/or programs to implement the example methods and/or operations described herein. These instructions may be stored in local memory 14, loaded in volatile memory 16, or in non-volatile memory 18.

The processor 12 may comprise one or more processors of various types suitable to the local application environment, and may include one or more of general-purpose computers, special-purpose computers, microprocessors, digital signal processors (DSPs), field-programmable gate arrays (FPGAs), application-specific integrated circuits (ASICs), and processors based on a multi-core processor architecture, as non-limiting examples. Examples of the processor 12 include one or more INTEL microprocessors, microcontrollers from the ARM and/or PICO families of microcontrollers, embedded soft/hard processors in one or more FPGAs. The local memory 14 may be any memory embedded in the processor 12 itself. The volatile memory 16 may be any kind of random-access memory (RAM). The non-volatile memory 18 may be any type of read-only memory (ROM).

The instructions executed by the processor 12 may comprise modeling or predictive routines, equations, algorithms, processes, applications, and/or other programs operable to perform example methods and/or operations described herein.

The processing device 10 may also comprise a bus 20 for communication with an interface circuit 22, which may comprise or be implemented via standard interfaces, including universal serial bus (USB), third-generation input/output (3GIO), peripheral component interconnect (PCI) Express, or any other known interfacing standard. This interface circuit 22 may in turn communicate with the input device 28, monitor display 30, and physiologic monitor 40. By way of non-limiting examples, the input device 28 may comprise a keyboard, the monitor display 30 may comprise a liquid crystal display (LCD), and the physiologic monitor 40 may comprise an EKG, the control device 40 may comprise a keyboard, and the display device 50 may comprise a liquid crystal display (LCD), all connected to the processing device 10 via interface circuit 22 and bus 20.

Some embodiments of the processing device 10 may comprise additional mass storage 24, representing memory capacity added via the bus 20. These may include disk drives, flash drives, and magnetic hard drives.

Some embodiments of the processing device 10 may comprise a network connection 26, which may be directly wired to the bus 20 (e.g., embedded Ethernet) or wired through the interface circuit 22 (e.g., USB wi-fi). The network connection 26 may utilize Ethernet, wi-fi, Bluetooth, cellular data, or any suitable communication protocol.

Some embodiments of the processing device 10 may omit one or more of these components, e.g., the internet connection 26 may be omitted for security reasons, or may be configured slightly differently, e.g., the mass storage 24 may be USB as well and thus connect through the interface circuit.

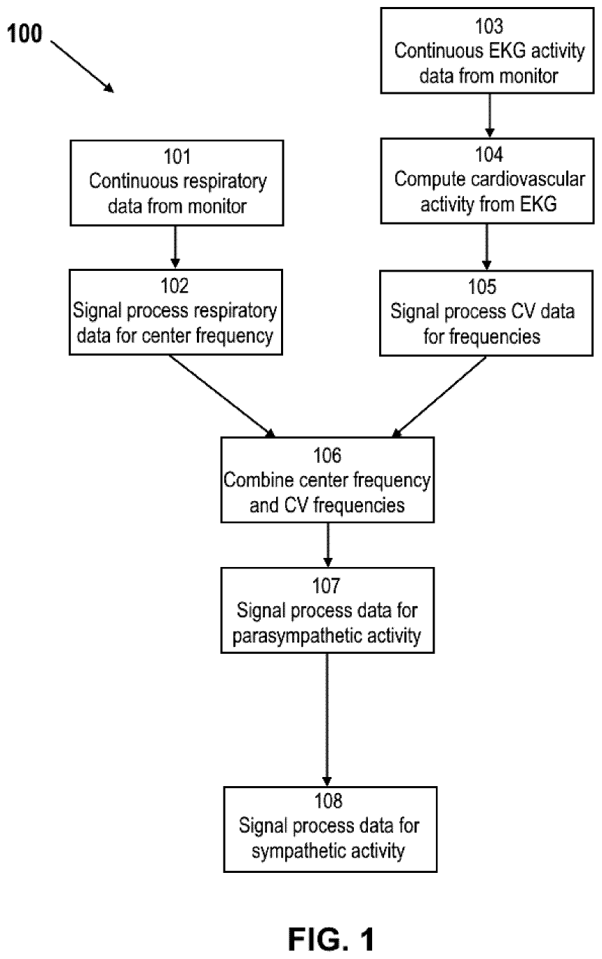

Documenting P&S dysfunction requires several steps to properly identify parasympathetic and sympathetic activity independently and simultaneously. Turning now to FIG. 1, a flowchart of the data collection method (100) is shown. Step 1 (101) collects real time continuous respiratory activity from an FDA-cleared physiologic monitor (shown as 40 in the previous figure). Step 2 (102) is to analyze the respiratory activity using one of a plethora of signal processing techniques to determine the center frequency of the respiratory activity over the time period of data collection. Step 3 (103) collects real time continuous EKG activity from a FDA cleared physiologic monitor. Step 4 (104) computes the EKG activity from the series of heartbeat intervals. Step 5 (105) analyzes the series of heartbeat intervals using one of a plethora of signal processing techniques to determine the frequencies of autonomic activity over the time period of data collection. Step 6 (106) is to combine said center frequency of the respiratory activity from Step 2 (102) and said frequencies of autonomic activity from Step 5 (105) and use said center frequency to isolate the Parasympathetic activity. Step 7 (107) is to compute parasympathetic activity over the time period of data collection using a plethora of signal processing techniques from Step 6 (106). Step 8 (108) is to compute the sympathetic activity over the time period of data collection using a signal processing techniques from the remaining sympathetic activity range from Step 7.

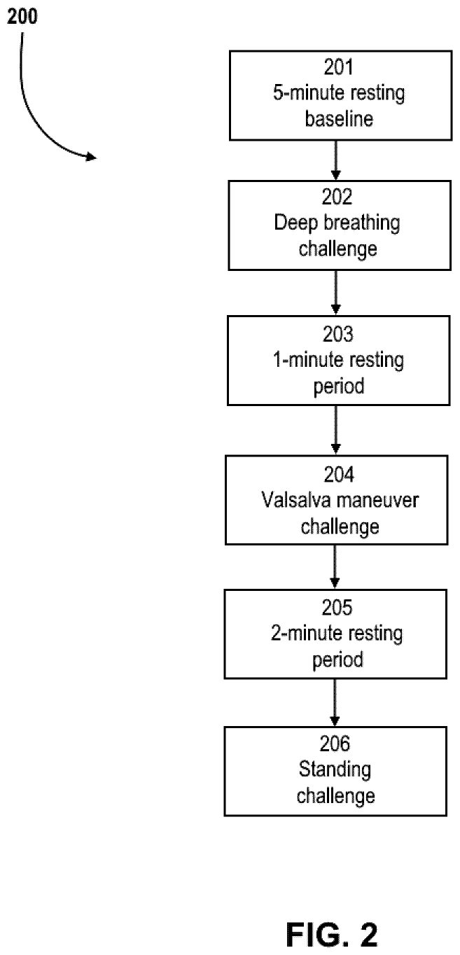

Turning now to FIG. 2, a broader diagnostic methodology (200) for the above sequence (100) is shown, in which the sequence of FIG. 1 is repeated, in the case of a chronic care setting, every four seconds during a two-to five-minute resting baseline (201). Next, the sequence of FIG. 1 is repeated every four seconds during a one-minute paced or deep breathing challenge (202). Next, the sequence is repeated every four seconds during a one-minute rest period (203). Next, the sequence is repeated every four seconds during a series of Valsalva maneuvers (204). In an embodiment, the series of Valsalva maneuvers may comprise the first Valsalva maneuver being 15 seconds in duration, followed by 20 seconds of breathing, followed by four shorter Valsalva maneuvers of 10 seconds in duration followed by 5 seconds of normal breathing. Next, the sequence is repeated every four seconds for a two-minute rest period (205). Finally, the sequence of FIG. 1 is repeated every four seconds for a five-minute or more “standing challenge” (206). This challenge may comprise standing from a supported seating position, sitting up from a supine position, or a positive head-up tilt (typically to 60°), followed by maintaining that standing, sitting, or head-up posture for the remainder of the five-minute or more period.

The sequence of FIG. 1 can also be applied in critical care settings, in which case any of the steps 1-8 are repeated as often as needed during the individual patient's stay.

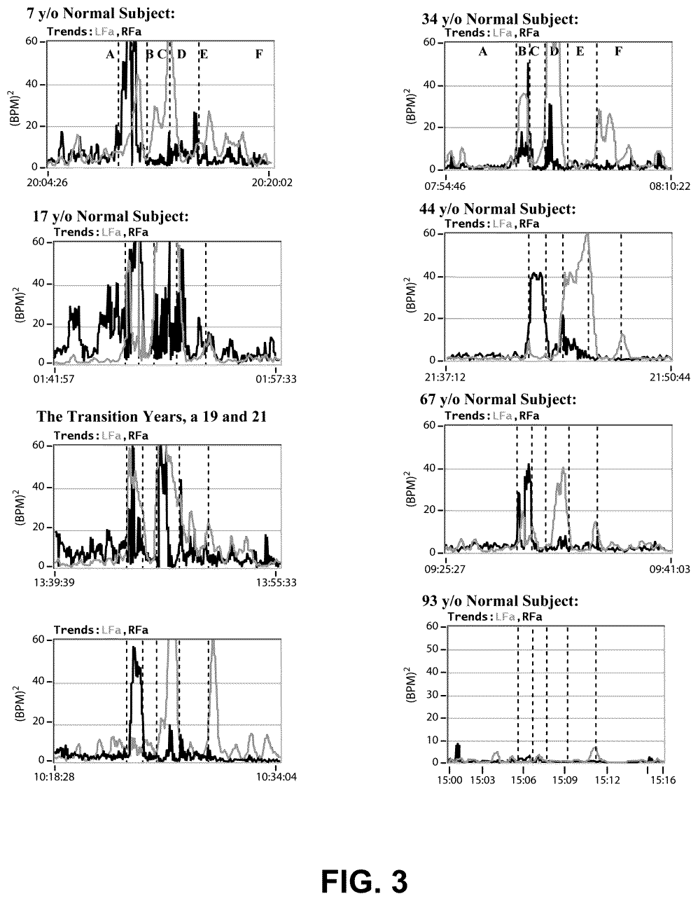

Turning to FIG. 3, trends plots of the “healthy” baselines for sympathetic activity (LFa) and parasympathetic activity (RFa) are shown over the course of several minutes' worth of data from various patients of various ages. As illustrated by FIG. 3, any finding of abnormal activity in either the sympathetic or parasympathetic system must be diagnosed relative to the base cohort.

Sympathovagal balance (SB) can be calculated based on (1) the ratio of sympathetic to parasympathetic activity as depicted on the trends plot; (2) the average of those ratios over the desired time period. “High,” “normal,” and “low” SB are defined as follows: h>1.1>n>0.4 for a geriatric patient or patient with a chronic disease, and h>3.0>n>0.9 for all other patients.

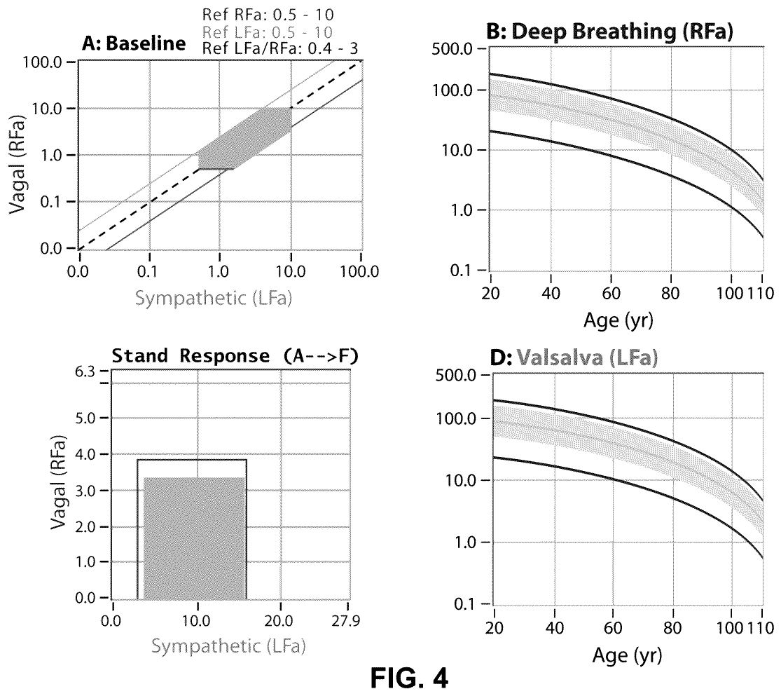

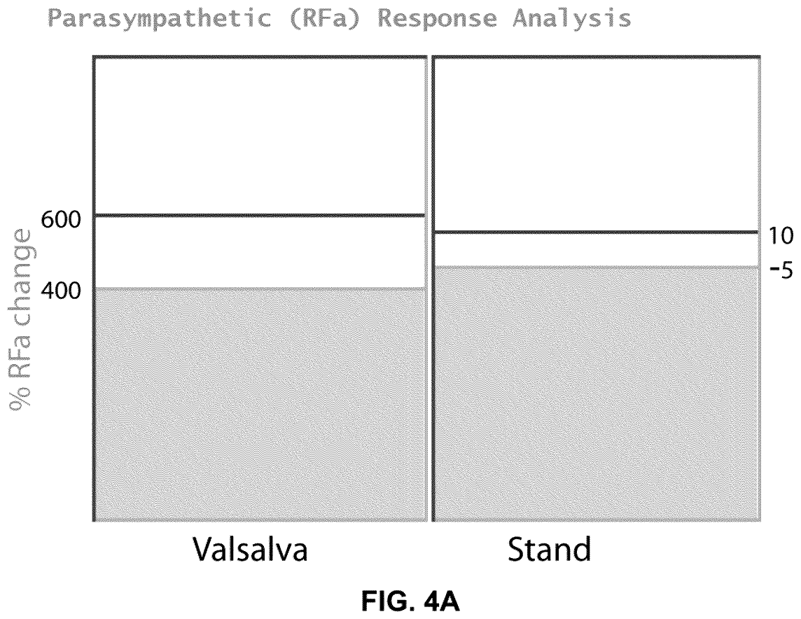

Turning to FIG. 4, several charts are shown indicating the “normal” ranges of sympathovagal balance for the various steps of FIG. 2. The baseline and standing challenge are judged by the ratio of sympathetic and parasympathetic activity during the challenge. Abnormal parasympathetic activity can be determined from an age-adjusted response to the deep breathing challenge, while abnormal sympathetic activity can be determined from an age-adjusted response curve to the Valsalva challenge.

Age adjustments are common in the art; exemplar age-adjustment curves for the deep breathing and Valsalva challenges can be found in Appendix I of “Autonomic Failure: A Textbook of Clinical Disorders of the Autonomic Nervous System” by Akinola, et al (Oxford Medical Publications, pp 194-5).

Because the autonomic nervous system controls every system in the body, there are numerous different medical or clinical conditions which can be indicated by the same autonomic dysfunctions depending on which organ system is the weakest at the moment. By applying the standards discussed herein to the tests laid out in FIGS. 1-2, these diagnoses can be differentiated by means of the relative sympathetic and parasympathetic activity levels.

Several disorders can be indicated from the computed SB value. For instance, advanced autonomic dysfunction (AAD) or diabetic autonomic neuropathy (DAN) is indicated if, during the baseline challenge (201), the sympathetic activity is below 0.5 bpm² or parasympathetic activity is between 0.1 bpm² and 0.5 bpm². Parasympathetic activity below 0.1 bpm² is indicative of cardiovascular autonomic neuropathy (CAN). These values, in conjunction with the “high” and “low” SB values, can indicate high morbidity risk (AAD or DAN with high/low SB) or mortality risk (CAN with high/low SB).

Parasympathetic excess (PE, aka vagal excitation) is computed from: (1) low SB; (2) an age-adjusted excessive increase in deep-breathing parasympathetic activity (202) over the resting parasympathetic response (201); (3) an increase in Valsalva parasympathetic activity (204) of over 600% (400-600% borderline) from the resting parasympathetic response (201); or (4) any increase in standing challenge parasympathetic activity (206) over the resting response (201). Since these values represent averages, an instantaneous PE is also determined. Instantaneous PE is determined as excessive Parasympathetic activity over a period of time from the Trends plot, whether or not the average falls within accepted normal ranges. An example of this can be found in FIG. 4A, showing the possible abnormal responses from the Valsalva challenge and standing challenge.

Sympathetic excess (SE, a beta-Adrenergic or Sympathetic response, aka hyperadrenergic response) is computed from either (1) high SB, as defined by a ratio of sympathetic activity to parasympathetic activity of 2.5:1 or greater; (2) an age adjusted excessive increase in Valsalva sympathetic activity (206) over the resting sympathetic response (201); or (3) an increase in standing challenge sympathetic activity of over 500% from the resting sympathetic response (206). As with the PE, these are averages, and an Instantaneous SE is also determined as excessive sympathetic activity over a period of time from the Trends plot, whether or not the average falls within accepted normal ranges.

Differentiating these two excesses can be crucial in differentiating between key diagnoses. For instance, PE is well-known to lead to daytime sleepiness, while SE is well known to lead to night time sleeplessness. P&S monitoring helps to differentiate the two causes. Sleep apnea is often comorbid with metabolic diseases such as diabetes and obesity. If a patient is diagnosed with sleep apnea in conjunction with any one of these, the patient is likely to have SE (among other dysfunctions). By treating the apnea alone, only a portion of the SE is addressed; leaving the patient at risk for major adverse cardiovascular events.

POTS and VVS are difficult to independently diagnose, as between 33-50% of all POTS patients are comorbid for VVS. Theoretically, the orthostatic hypotension can be indicated by sympathetic withdrawal (SW, and alpha-Adrenergic or Sympathetic response), while the vasovagal syncope can be independently diagnosed via parasympathetic excess with SE. However, since the tachycardia of POTS is known to be a compensatory mechanism for the orthostatic dysfunction, and presents itself as sympathetic excess (SE), this can have the effect of masking the signal.

SE can be diagnosed via what are known as “syncope spikes” during the standing test, or recurring sympathetic peaks after the initial spike upon standing.

Furthermore, many gastrointestinal disorders start as motility disorders. The parasympathetic system controls the GI tract by controlling motility; many GI disorders involve the same set of symptoms, but one may be due to PE and the other insufficient RFa. Differentiating the two facilitates diagnosis and treatment.

Additionally, the parasympathetic nervous system is the link to the pleasure centers of the brain, playing a crucial role in addiction. PE drives the individual to whatever behavior is associated with the pleasure center; if rehabilitation does not diagnose and address PE, addicts will be more prone to relapsing.

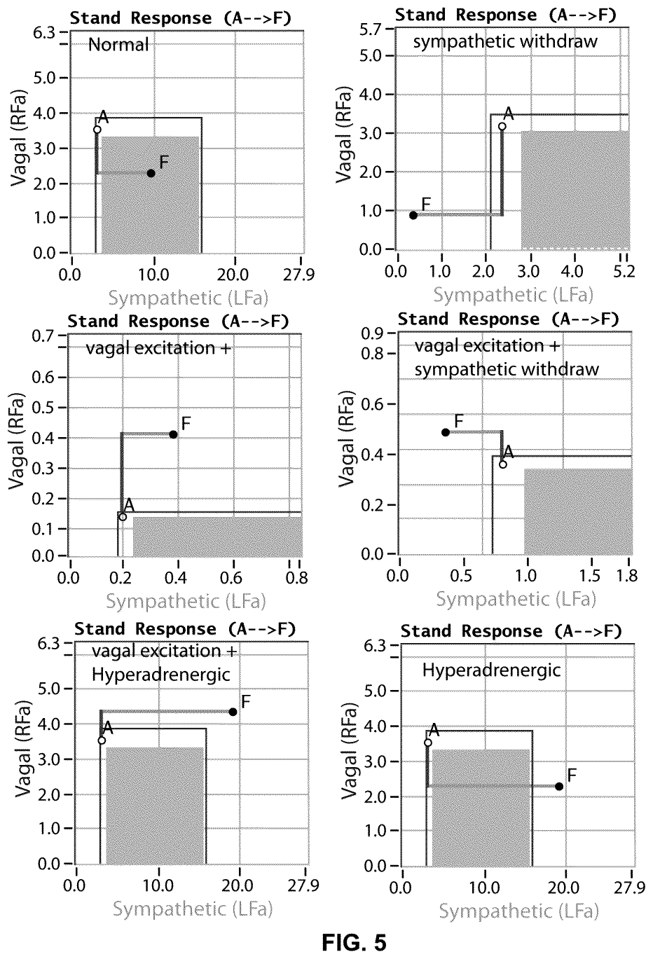

Turning now to FIG. 5, a series of graphical outputs are shown which indicate various exemplar patients' responses to the full series of challenges, which are plotted on a chart in which the sympathetic and parasympathetic activity is shown as two axes, with the patient's sympathovagal balance indicated relative to a baseline square range (two are shown in these examples: a solid-colored baseline and an outline for “borderline” values; other embodiments may only utilize a single boundary). The first patient's responses are normal and indicated by the balance remaining within the square. Abnormalities in this embodiment are indicated by the balance ending outside the borderline square. (In an embodiment, these may also be indicated directly on the screen via text.)

Alpha-sympathetic withdrawal (SW) is computed from any decrease in standing challenge sympathetic activity (206) compared with the resting sympathetic response (201), as shown in the second and fourth graphs of FIG. 5. SW may be masked by PE or SE, and must be confirmed by an abnormal BP or HR or an instantaneous SE response to standing challenge compared with resting response.

Heart failure may be caused by prolonged or untreated SW, which leads to low diastolic pressures, poor cardiac perfusion, poor cerebral perfusion, and finally increased systolic pressure. Once the difference between these pressures exceeds 80 mm Hg, heart failure is indicated; treating SW can thus help reduce the risk of heart failure.

The third, fourth, and fifth graphs of FIG. 5 indicate PE (i.e., vagal excitation), while the fifth and sixth graphs of FIG. 5 indicate SE (i.e., hyperadrenergic responses).

There are at least four P&S dysfunctions which lead to secondary hypertension as a compensatory mechanism for other symptoms or disorders. For example, a drop in BP upon standing (known as orthostatic hypotension) may lead to increased resting BP so that when BP falls upon standing, the brain remains marginally perfused.

An abnormal HR response to stand would be an increase over 20 bpm indicating POTS. This is a three-fold abnormality by which time most patients have experienced a multitude of additional co-morbidities. With additional P&S information, a pre-clinical POTS indication is possible to diagnose earlier and treat earlier before additional co-morbidities present. Similarly, a 30/20 mmHg increase in BP indicates orthostatic hypertension, or 20 mmHg systolic drop OR 10 mmHg diastolic drop indicates orthostatic hypotension. Again, these are three-fold abnormalities by which time most patients have experienced a multitude of additional co-morbidities. With additional P&S information, a pre-clinical POTS indication is possible to diagnose earlier and treat earlier before additional co-morbidities present. (In addition, orthostatic intolerance can be indicated from a lack of 10/5 mmHg increase in BP during the standing challenge.)

For all P&S dysfunctions co-morbid with hypertension, treating the hypertension as a primary disorder can exacerbate both it and the underlying P&S dysfunction. Documenting the P&S dysfunction helps to protect against the life threatening effects of hypertension, while treating the P&S dysfunction often relieves the hypertension as an effect.

These findings in turn can indicate several other disorders. Small Fiber Disease (SFD) risk is indicated by either or both of (1) an abnormally low, age adjusted increase in deep breathing parasympathetic activity over the resting parasympathetic response, or (2) an abnormally low, age adjusted increase in Valsalva challenge sympathetic activity over resting sympathetic response.

Falling risk can be computed based on the severity of (1) SW with abnormal changes in BP or pulse during the standing challenge (clinically known as orthostatic dysfunction); (2) PE with standing challenge SE (clinically known as vasovagal syncope); or (3) standing challenge SE with abnormally low (5 bpm or less) pulse increase from resting (clinically known as neurogenic syncope).

Major depression disorder (MDD) and other depression-related disorders can be computed based on the severity of low SB (0.9). Depression/Anxiety disorders that are marginally responsive to treatment (including requiring more treatment) may involve PE and SW. Risk of re-hospitalization is computed based on severity of abnormal SB. Risk of repeat addiction (including Opioid addiction) is computed based on severity of PE after rehabilitation.

Treating P&S (autonomic) dysfunction depends on the P&S Dysfunction(s) documented and patient history. When treating P&S dysfunctions, PE and SW are considered primary autonomic dysfunctions, with all other P&S dysfunctions are secondary to these two, if documented (if PE/SW are not documented, then those dysfunctions may be primary). Also, in treating P&S dysfunctions, very low dose, very slow therapy overtime is required. P&S dysfunctions that are treated with high doses or multiple agents in the hopes of faster recovery times almost always cause more P&S dysfunction.

PE may be treated pharmaceutically with low dose (homeostatic) anti-cholinergic therapy, or non-pharmaceutically with low and slow exercise over a six month or more period of time. There are a plethora of other PE therapies, including GI motility medications, bladder dysfunction medications, breathing techniques, stress reduction and meditation techniques, yoga, prayer, etc.

SE may be treated pharmaceutically with beta blockers or anti-hypertensives or some other sympatholytic dependent on patient history. However, if SE presents with PE, treating PE typically relieves SE without further intervention. After PE is relieved, any SE remaining may be treated directly and more aggressively. However, treating SE without treating PE will exacerbate both and most likely make the patient more difficult to manage. Under certain conditions Vagal Nerve Stimulators may be employed to help balance the P&S nervous systems.

SW may be treated pharmaceutically with low dose (homeostatic) oral vasoactive therapy, augmented by mineralocorticoids to increase blood volume, or by decreasing oral diuretics. SW may also be treated non-pharmaceutically with r-Alpha-Lipoic Acid. Regardless of mode of therapy, SW should also be treated with proper daily hydration, electrolytes depending on patient history, and compression garments.

Abnormal SB is treated by normalizing SB. If SB is low, indicating PE, the above PE treatment should be followed. If SB is high, indicating resting SE with PE not indicated, treat with sympatholytics (history dependent).

Normalizing SB can reduce the high morbidity risks of AAD, DAN, and CAN. If AAD, DAN, or CAN is documented alongside low parasympathetic activity, under certain conditions vagal nerve stimulators specific for the symptoms or organ system affected may be employed to help normalize SB and thereby treat the low Parasympathetic activity and co-morbid AAD, DAN, or CAN.

Several cardiological conditions can be differentiated by means of this method. Atrial fibrillation involves the parasympathetic system approximately ⅔ of the time, with the remaining third split between sympathetically mediated and cardiogenic atrial fibrillation. Thus, up to 5/6 of patients experiencing atrial fibrillations may benefit from a treatment plan focused on restoring sympathovagal balance.

Normalizing PE also has the potential to treat mental disorders. There are at least four disorders which cause anxiety-like symptoms (general anxiety, ADD, depression, and PTSD), all of which are associated with P&S dysfunction, which in turn is associated with inadequate cerebral perfusion (blood flow to the brain). By treating the the P&S dysfunctions, proper cerebral perfusion can be restored, and the symptoms of the disorders can be relieved.

Numerous other conditions can also benefit from the consistent documentation of sympathetic and parasympathetic activity. Pain management can permit differentiation between SE-mediated and PE-mediated pain, enabling earlier identification of CRPS. Diabetes can be treated more easily if the autonomic neuropathy is documented, helping to minimize morbidity. P&S imbalance can lead to problems with hormone imbalance, pregnancy risks, and general quality of life. Additionally, since many workman's compensation claims are based on P&S dysfunction, documenting this activity before and after an incident can ensure that legitimate claims are not overlooked, or spurious claims not accepted.

1. A method of differentiating sympathetic and parasympathetic disorder risk levels in a monitoring device, comprising:
Obtaining, in a plurality of stages, pulse data and respiratory data from a monitoring device connected to a patient, each stage corresponding to a level of exertion by the patient, over a period of time;
Computing, using signal analysis and the pulse data and respiratory data of the patient, sympathetic activity and parasympathetic activity for each stage;
Computing, based on the sympathetic activity and parasympathetic activity, a sympathovagal balance for each stage;
Detecting, based on a threshold of the sympathetic activity, parasympathetic activity, sympathovagal balance, or a combination thereof, whether the patient exhibits beta-sympathetic excess;
Detecting, based on a threshold of the sympathetic activity, parasympathetic activity, sympathovagal balance, or a combination thereof, whether the patient exhibits parasympathetic excess;
Detecting, based on a threshold of the sympathetic activity, parasympathetic activity, sympathovagal balance, or a combination thereof, whether the patient exhibits alpha-sympathetic withdrawal; and
Generating, on a display, a chart for the patient and health care provider, wherein the sympathetic activity is displayed on a first axis, the parasympathetic activity displayed on a second axis, and wherein the presence of alpha-sympathetic withdrawal, beta-sympathetic excess, or parasympathetic excess is indicated if detected.

2. The method of claim 1, wherein the step of obtaining the pulse data and respiratory data comprises the stages of resting, deep breathing, Valsalva maneuver, and standing.

3. The method of claim 2, wherein the step of detecting parasympathetic excess comprises one or more of the following conditions:
a sympathovagal balance under 0.9, or 0.4 for geriatric and chronically ill patients;
an age-adjusted excessive increase in parasympathetic activity during the deep breathing, compared to resting;
an age adjusted increase in parasympathetic activity of over 600% during the Valsalva maneuver, compared to resting; and
any increase in parasympathetic activity during standing, compared to resting.

4. The method of claim 2, wherein the step of detecting sympathetic excess comprises one or more of the following conditions:
a sympathovagal balance over 3.0, or 1.1 for geriatric and chronically ill patients;
an age-adjusted excessive increase in sympathetic activity during the Valsalva maneuver, compared to resting; and
an increase in sympathetic activity during standing of over 500%, compared to resting.

6. The method of claim 2, wherein the step of detecting alpha-sympathetic withdrawal comprises the condition wherein there is a decrease in sympathetic activity during standing, compared to resting.

7. The method of claim 1, comprising the additional step of calculating a falling risk, comprising one or more of the following conditions:
if the patient indicates sympathetic excess, an increase in pulse of less than 5 bpm during standing, compared to resting;
if the patient indicates beta-sympathetic excess during the standing challenge, and also alpha-sympathetic withdrawal, an abnormal increase in pulse or decrease in blood pressure during the standing challenge, compared to resting; and
if the patient indicates sympathetic excess during standing, and any parasympathetic excess during the other stages.

8. The method of claim 1, comprising the additional step of calculating a small fiber disease risk, comprising one or more of the following conditions:
an age-adjusted abnormally low increase in parasympathetic activity during the deep breathing, compared to resting; and
an age-adjusted abnormally low increase in sympathetic activity during the Valsalva maneuver, compared to resting.
